(19) United States
(12) Patent Application Publication
(10) Pub. No.: US 2005/0038048 A1
(43) Pub. Date: Feb. 17, 2005

(54) Use of 7h-pyrrollo{2,3-d}pyrimidine derivatives in the treatment of solid tumor diseases

(76) Inventors: Howard Ashley Ball, Kendall Park, NJ (US); Pamela Sarah Cohen, Tenafly, NJ (US); Lucy Lee, Parsippany, NJ (US); Christina Portrude Ravera, Chatham, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/493,787

(22) PCT Filed: Oct. 28, 2002
(86) PCT No.: PCT/EP02/12024

Related U.S. Application Data
(60) Provisional application No. 60/340,923, filed on Oct. 29, 2001.
(60) Provisional application No. 60/361,655, filed on Mar. 5, 2002.
(60) Provisional application No. 60/379,365, filed on May 9, 2002.

(51) Int. Cl. A61K 31/519

(52) U.S. Cl. 514/265.1

(57) ABSTRACT

Patients suffering from a solid tumor disease selected from carcinoma of the bladder, renal carcinoma, squamous cell carcinoma of the skin, head and neck cancer, especially squamous cell head and neck cancer, lung cancer, especially non small cell lung cancer (NSCLC), tumors of the gastrointestinal tract, glioma and mesothelioma or metastases of such solid tumor diseases are treated with a 7H-pyrrolo[2,3-d]pyrimidine derivative.

This invention relates to a method for the treatment of patients suffering from a solid tumor disease selected from carcinoma of the bladder, renal carcinoma, squamous cell carcinoma of the skin, head and neck cancer, especially squamous cell head and neck cancer, lung cancer, especially non small cell lung cancer (NSCLC), tumors of the gastrointestinal tract, glioma and mesothelioma comprising administering a 7H-pyrrolo[2,3-d]pyrimidine derivative, or a pharmaceutically acceptable salt thereof, in particular using an improved regimen for the administration of such 7H-pyrrolo[2,3-d]pyrimidine derivative.

7H-pyrrolo[2,3-d]pyrimidine derivatives useful for treating tumor diseases and other conditions are, e.g., disclosed in U.S. Pat. No. 6,140,332, which is here incorporated by reference in its entirety. Such 7H-pyrrolo[2,3-d]pyrimidine derivatives are described in such patent to be useful for the treatment of benign or malignant tumours being capable of effecting tumour regression and of preventing the formation of tumour metastases and the growth of micrometastases. According to such patent such compounds can be used especially in the case of epidermal hyperproliferation (psoriasis), in the treatment of neoplasias of epithelial character, e.g. mammary carcinomas, and in leukaemias.

Furthermore, U.S. Pat. No. 6,140,332 discloses that the 7H-pyrrolo[2,3-d]pyrimidine derivatives are administered in the case of an individual having a body weight of about 70 kg at a daily dose from approximately 0.1 grams to approximately 5 grams, preferably from about 0.5 grams to 2 grams. It is not suggested that the 7H-pyrrolo[2,3-d]pyrimidine derivative should be administered on alternate days.

Surprisingly, it was now found that 7H-pyrrolo[2,3-d]pyrimidine derivatives are suitable for the treatment of the solid tumor diseases mentioned herein. Thus, the present invention relates to the use of a 7H-pyrrolo[2,3-d]pyrimidine derivative, or a pharmaceutically acceptable salt thereof, for the manufacture of a medicament for the treatment of carcinoma of the bladder, renal carcinoma, squamous cell carcinoma of the skin, head and neck cancer, especially squamous cell head and neck cancer, lung cancer, especially non small cell lung cancer (NSCLC), tumors of the gastrointestinal tract, glioma or mesothelioma.

Furthermore, the present invention relates to a method for the treatment of patients suffering from a solid tumor disease selected from renal carcinoma, squamous cell carcinoma of the skin, head and neck cancer, especially squamous cell head and neck cancer, lung cancer, especially NSCLC, tumors of the gastrointestinal tract, glioma and mesothelioma comprising administering a 7H-pyrrolo[2,3-d]pyrimidine derivative, or a pharmaceutically acceptable salt thereof, in particular using an improved regimen for the administration of such 7H-pyrrolo[2,3-d]pyrimidine derivative as described herein.

The present invention further relates to a method of inhibiting metastatic growth in a patient with a solid tumor disease as defined herein which comprises administering a pharmaceutically effective amount of a 7H-pyrrolo[2,3-d]pyrimidine derivative or a pharmaceutically acceptable salt thereof, to the patient, in particular using an improved regimen for the administration of such 7H-pyrrolo[2,3-d]pyrimidine derivative as described herein.

In the present invention, the compound (R)-6-(4-hydroxy-phenyl)-4-[(1-phenyl-ethyl)-amino]-7H-pyrrolo[2,3-d]pyrimidine, or a pharmaceutically acceptable salt therof, is the preferred 7H-pyrrolo[2,3-d]pyrimidine derivative, which compound is described in Example 39 of WO 97/02266. The compound is also known in the art as “PKI166” or “CGP 75166”.

Another aspect of the present invention is the use of 7H-pyrrolo[2,3-d]pyrimidine derivative of formula I 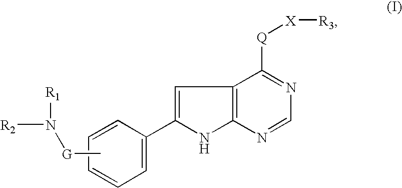
wherein
R₁ and R₂ are each independently of the other hydrogen, unsubstituted or substituted alkyl or cycloalkyl, a heterocyclic radical bonded via a ring carbon atom, or a radical of the formula R₄—Y—(C═Z)— wherein R₄ is unsubstituted, mono- or disubstituted amino or a heterocyclic radical, Y is either not present or lower alkyl and Z is oxygen, sulfur or imino, with the proviso that R₁ and R₂ are not both hydrogen; or
R₁ and R₂ together with the nitrogen atom to which they are attached form a heterocyclic radical;
R₃ is a heterocyclic radical or an unsubstituted or substituted aromatic radical;
G is C₁-C₇-alkylene, —C(═O)—, or C₁-C₆-alkylene-C(═O)— wherein the carbonyl group is attached to the NR₁R₂ moiety;
Q is —NH— or —O—, with the proviso that Q is —O— if G is —C(═O)— or C₁-C₆-alkylene-C(═O)—; and
X is either not present or C₁-C₇-alkylene, with the proviso that a heterocyclic radical R₃ is bonded via a ring carbon atom if X is not present;
or a salt of the said compounds,
   for the treatment of carcinoma of the bladder, renal carcinoma, squamous cell carcinoma of the skin, tumors of the gastrointestinal tract, mesothelioma, esophageal tumors, stomach cancer, small-bowel tumors and large-bowel tumors such as polyps of the colon and rectum, and anorectal cancer.

A further aspect of the present invention is a method of inhibiting metastatic growth in a patient with a solid tumor disease selected from carcinoma of the bladder, renal carcinoma, squamous cell carcinoma of the skin, tumors of the gastrointestinal tract, mesothelioma, esophageal tumors, stomach cancer, small-bowel tumors and large-bowel tumors such as polyps of the colon and rectum, and anorectal cancer which comprises administering to the patent a pharmaceutically effective amount of a 7H-pyrrolo[2,3-d]pyrimidine derivative of formula I wherein R₁ and R₂ are each independently of the other hydrogen, unsubstituted or substituted alkyl or cycloalkyl, a heterocyclic radical bonded via a ring carbon atom, or a radical of the formula R₄—Y—(C═Z)— wherein R₄ is unsubstituted, mono- or disubstituted amino or a heterocyclic radical, Y is either not present or lower alkyl and Z is oxygen, sulfur or imino, with the proviso that R₁ and R₂ are not both hydrogen; or R₁ and R₂ together with the nitrogen atom to which they are attached form a heterocyclic radical; R₃ is a heterocyclic radical or an unsubstituted or substituted aromatic radical; G is C₁-C₇-alkylene, —C(═O)—, or C₁-C₆-alkylene-C(═O)— wherein the carbonyl group is attached to the NR₁R₂ moiety; Q is —NH— or —O—, with the proviso that Q is —O— if G is —C(═O)— or C₁-C₆-alkylene-C(═O)—; and X is either not present or C₁-C₇-alkylene, with the proviso that a heterocyclic radical R₃ is bonded via a ring carbon atom if X is not present; or a salt of the said compounds, for the treatment of carcinoma of the bladder, renal carcinoma, squamous cell carcinoma of the skin, tumors of the gastrointestinal tract, mesothelioma, esophageal tumors, stomach cancer, small-bowel tumors and large-bowel tumors such as polyps of the colon and rectum, and anorectal cancer.

The general terms used hereinbefore and hereinafter preferably have within the context of this disclosure the following meanings, unless otherwise indicated:

As used herein, the expression “week” means seven consecutive days. Thus, a three week period is twenty-one consecutive days starting on any day of the calendar week. The day that the first dose is given is considered to be the first day of the week. Any discussion using calendar weeks is intended to be for illustrative purposes only.

As used herein, the expression “mesothelioma” means a malignant tumor derived from mesothelial tissue (peritoneum, pleura, pericardium).

As used herein, the expression “glioma” preferably includes all primary intrinsic neoplasms of the brain and spinal cord, e.g. astrocytomas, ependymomas, neurocytomas or meningiomas.

The term “tumors of the gastrointestinal tract” as used herein, includes, but is not limited to esophageal tumors, stomach cancer, small-bowel tumors and large-bowel tumors such as polyps of the colon and rectum, colorectal cancer and anorectal cancer.

As used herein, the expression “partial response” means a greater than or equal to 50% reduction in measurable or evaluable disease in the absence of progression in any particular disease site.

As used herein, the expression “stable disease” means a less than 50% decrease or less than 25% increase in measurable or evaluable disease.

Asymmetric carbon atoms of a compound of formula I that are optionally present may exist in the (R), (S) or (R,S) configuration, preferably in the (R) or (S) configuration. Substituents at a double bond or a ring may be present in cis- (═Z—) or trans (═E—) form. The compounds may thus be present as mixtures of isomers or preferably as pure isomers.

Preferably alkyl contains up to 20 carbon atoms and is most preferably lower alkyl.

The prefix “lower” denotes a radical having up to and including a maximum of 7, especially up to and including a maximum of 4 carbon atoms, the radicals in question being either unbranched or branched with single or multiple branching.

Lower alkyl is, for example, methyl, ethyl, n-propyl, isopropyl, n-butyl, isobutyl, sec-butyl, tert-butyl, n-pentyl, isopentyl, neopentyl, n-hexyl or n-heptyl.

Alkyl R₁ and R₂ independently of each other are preferably methyl, ethyl, isopropyl or tert-butyl, especially methyl or ethyl.

Lower alkyl Y is preferably methyl, ethyl or propyl.

Lower alkoxy is for example ethoxy or methoxy, especially methoxy.

Substituted alkyl is preferably lower alkyl as defined above where one or more, preferably one, substituents may be present, such as e.g. amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio, halogen or a heterocyclic radical.

Substituted alkyl R₁ and R₂ are independently of each other preferably hydroxy-lower alkyl, N,N-di-lower alkylamino-lower alkyl or morpholinyl-lower alkyl.

Preferably unsubstituted or substituted cycloalkyl R₁ or R₂ contains from 3 up to 20 carbon atoms and is especially unsubstituted or also substituted C₃-C₆ cycloalkyl wherein the substituents are selected from e.g. unsubstituted or substituted lower alkyl, amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio, halogen or a heterocyclic radical.

Mono- or disubstituted amino is amino substituted by one or two radicals selected independently of one another from e.g. unsubstituted or substituted lower alkyl.

Disubstituted amino R₄ is preferably N,N-di-lower alkylamino, especially N,N-dimethylamino or N,N-diethylamino.

A heterocyclic radical contains especially up to 20 carbon atoms and is preferably a saturated or unsaturated monocyclic radical having from 4 or 8 ring members and from 1 to 3 heteroatoms which are preferably selected from nitrogen, oxygen and sulfur, or a bi- or tri-cyclic radical wherein, for example, one or two carbocyclic radicals, such as e.g. benzene radicals, are annellated (fused) to the mentioned monocyclic radical. If a heterocyclic radical contains a fused carbocyclic radical then the heterocyclic radical may also be attached to the rest of the molecule of formula I via a ring atom of the fused carbocyclic radical. The heterocyclic radical (including the fused carbocyclic radical(s) if present) is optionally substituted by one or more, preferably by one or two, radicals such as e.g. unsubstituted or substituted lower alkyl, amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio, or halogen.

Most preferably a heterocyclic radical is pyrrolidinyl, piperidyl, lower alkyl-piperazinyl, di-lower alkyl-piperazinyl, morpholinyl, tetrahydropyranyl, pyridyl, pyridyl substituted by hydroxy or lower alkoxy, or benzodioxolyl, especially pyrrolidinyl, piperidyl, lower alkyl-piperazinyl, di-lower alkyl-piperazinyl or morpholinyl.

A heterocyclic radical R₁ or R₂ is as defined above for a heterocyclic radical with the proviso that it is bonded to the rest of the molecule of formula I via a ring carbon atom. Preferably a heterocyclic radical R₁ or R₂ is lower alkyl-piperazinyl or especially preferred tetrahydropyranyl. If one of the two radicals R₁ and R₂ represents a heterocyclic radical, the other is preferably hydrogen.

A heterocyclic radical R₃ is as defined above for a heterocyclic radical with the proviso that it is bonded to Q via a ring carbon atom if X is not present. Preferably a heterocyclic radical R₃ is benzodioxolyl, pyridyl substituted by hydroxy or lower alkoxy, or especially preferred indolyl substituted by halogen and lower alkyl. If R₃ is pyridyl substituted by hydroxy then the hydroxy group is preferably attached to a ring carbon atom adjacent to the ring nitrogen atom.

A heterocyclic radical R₄ is as defined above for a heterocyclic radical and is preferably pyrrolidinyl, piperidyl, lower alkyl-piperazinyl, morpholinyl or pyridyl.

If R₁ and R₂ together with the nitrogen atom to which they are attached form a heterocyclic radical, the heterocyclic radical is as defined above for a heterocyclic radical and represents preferably pyrrolidinyl, piperidyl, lower alkyl-piperazinyl, di-lower alkyl-piperazinyl or morpholinyl.

An unsubstituted or substituted aromatic radical R₃ has up to 20 carbon atoms and is unsubstituted or substituted, for example in each case unsubstituted or substituted phenyl. Preferably an unsubstituted aromatic radical R₃ is phenyl. A substituted aromatic radical R₃ is preferably phenyl substituted by one or more substituents selected independently of one another from the group consisting of unsubstituted or substituted lower alkyl, amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio and halogen. Most preferably a substituted aromatic radical R₃ is phenyl substituted by one or more radicals selected independently of one another from the group consisting of lower alkyl, amino, hydroxy, lower alkoxy, halogen and benzyloxy.

Halogen is primarily fluoro, chloro, bromo or iodo, especially fluoro, chloro or bromo.

C₁-C₇-alkylene may be branched or unbranched and is in particular C₁-C₃-alkylene.

C₁-C₇-alkylene G is preferably C₁-C₃-alkylene, most preferably methylene (—CH₂—).

If G is not C₁-C₇-alkylene it preferably represents —C(═O)—.

C₁C₇-alkylene X is preferably C₁-C₃-alkylene, most preferably methylene (—CH₂—) or ethan-1,1-diyl (—CH(CH₃)—).

Q is preferably —NH—.

Z is preferably oxygen or sulfur, most preferably oxygen.

Salts are especially the pharmaceutically acceptable salts of compounds of formula I.

Special preference is given to a compound of formula I, wherein
R₁ and R₂ are each independently of the other hydrogen, lower alkyl, hydroxy-lower alkyl, or a radical of the formula R₄—Y—(C═Z)— wherein R₄ is di-lower alkylamino, pyrrolidinyl, piperidyl, lower alkyl-piperazinyl, morpholinyl or pyridyl, Y is either not present or lower alkyl and Z is oxygen, with the proviso that R₁ and R₂ are not both hydrogen; or
R₁ and R₂ together with the nitrogen atom to which they are attached form a radical selected from the group consisting of pyrrolidinyl, piperidyl, lower alkyl-piperazinyl, di-lower alkyl-piperazinyl and morpholinyl;
R₃ is phenyl, benzodioxolyl, pyridyl substituted by hydroxy or lower alkoxy, or phenyl substituted by one or more radicals selected independently of one another from the group consisting of lower alkyl, hydroxy, lower alkoxy, halogen and benzyloxy;
G is —CH₂—;
Q is —NH—; and
X is either not present, —CH₂— or —CH(CH₃)—, with the proviso that substituted pyridyl R₃ is bonded via a ring carbon atom if X is not present;
or a salt thereof.

The compounds of formula I or salts thereof are prepared in accordance with processes known per se (see also EP 682 027, WO 97/02266, WO 97/27199 and WO 98/07726), though not previously described for the manufacture of the compounds of the formula I, especially whereby in order to prepare a compound of formula I, wherein G is C₁-C₇-alkylene and wherein R₁ and R₂ are each independently of the other hydrogen, unsubstituted or substituted alkyl or cycloalkyl, or a heterocyclic radical bonded via a ring carbon atom, with the proviso that R₁ and R₂ are not both hydrogen, or wherein R₁ and R₂ together with the nitrogen atom to which they are attached form a heterocyclic radical, a compound of the formula II 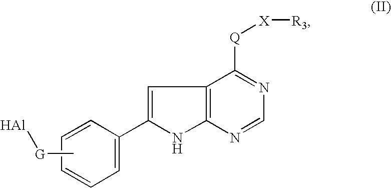
wherein Hal is halogen, G is C₁-C₇-alkylene and R₃, Q and X have the meanings as defined for a compound of formula I, is reacted with a compound of the formula III 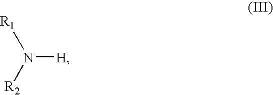
wherein R₁ and R₂ are each independently of the other hydrogen, unsubstituted or substituted alkyl or cycloalkyl, or a heterocyclic radical bonded via a ring carbon atom, with the proviso that R₁ and R₂ are not both hydrogen, or wherein R₁ and R₂ together with the nitrogen atom to which they are attached form a heterocyclic radical;
whereby functional groups which are present in the starting compounds of processes a) to d) and are not intended to take part in the reaction, are present in protected form if necessary, and protecting groups that are present are cleaved, whereby the said starting compounds may also exist in the form of salts provided that a salt-forming group is present and a reaction in salt form is possible;
and, if so desired, a compound of formula I thus obtained is converted into another compound of formula I, a free compound of formula I is converted into a salt, an obtained salt of a compound of formula I is converted into the free compound or another salt, and/or a mixture of isomeric compounds of formula I is separated into the individual isomers.

### Description of the Process Variants

The reaction between a compound of formula II and a compound of formula III preferably takes place in a suitable inert solvent, especially N,N-dimethylformamide, in the presence of a base such as potassium carbonate, at temperatures from room temperature (RT) to 100° C. Alternatively, the reaction between a compound of formula II and a compound of formula III takes place in a suitable solvent, e.g. lower alcohols, such as ethanol, in the presence of for example a suitable catalyst such as NaI, preferably at the reflux temperature of the solvent employed. In a compound of formula II, Hal is preferably chloro.

### Additional Process Steps

In the additional process steps, carried out as desired, functional groups of the starting compounds which should not take part in the reaction may be present in unprotected form or may be protected for example by one or more protecting groups. The protecting groups are then wholly or partly removed according to one of the known methods.

Protecting groups, and the manner in which they are introduced and removed are described, for example, in “Protective Groups in Organic Chemistry”, Plenum Press, London, New York 1973, and in “Methoden der organischen Chemie”, Houben-Weyl, 4th edition, Vol. 15/1, Georg-Thieme-Verlag, Stuttgart 1974 and in Theodora W. Greene, “Protective Groups in Organic Synthesis”, John Wiley & Sons, New York 1981. A characteristic of protecting groups is that they can be removed readily, i.e. without the occurrence of undesired secondary reactions, for example by solvolysis, reduction, photolysis or alternatively under physiological conditions.

The end products of formula I may however also contain substituents that can also be used as protecting groups in starting materials for the preparation of other end products of formula I. Thus, within the scope of this text, only a readily removable group that is not a constituent of the particular desired end product of formula I is designated a “protecting group”, unless the context indicates otherwise.

### General Process Conditions

All process steps described here can be carried out under known reaction conditions, preferably under those specifically mentioned, in the absence of or usually in the presence of solvents or diluents, preferably those that are inert to the reagents used and able to dissolve them, in the absence or presence of catalysts, condensing agents or neutralising agents, for example ion exchangers, typically cation exchangers, for example in the H⁺ form, depending on the type of reaction and/or reactants at reduced, normal, or elevated temperature, for example in the range from −100° C. to about 190° C., preferably from about −80° C. to about 150° C., for example at −80 to −60° C., at RT, at −20 to 40° C., at 0 to 100° C. or at the boiling point of the solvent used, under atmospheric pressure or in a closed vessel, if need be under pressure, and/or in an inert, for example an argon or nitrogen, atmosphere.

### Starting Materials

The starting materials used in the above described processes a) to b) are known, capable of being prepared according to known processes (see also EP 682 027, WO 97/02266, WO 97/27199 and WO 98/07726), or commercially obtainable; in particular, they can be prepared using processes as described in the Examples.

In the preparation of starting materials, existing functional groups which do not participate in the reaction should, if necessary, be protected. Preferred protecting groups, their introduction and their removal are described above or in the Examples. Where the term starting materials is used hereinbefore and hereinafter, the salts thereof are always included, insofar as reasonable and possible.

A compound of formula II can be prepared for example by reacting a compound of formula VII 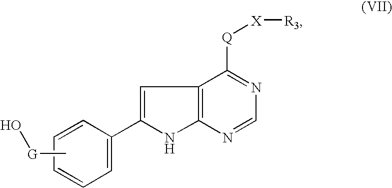
wherein G is C₁-C₇-alkylene and R₃, Q and X have the meanings as defined for a compound of formula I, with e.g. thionyl halogenide, preferably thionyl choride, in the presence or absence of pyridine, in an inert solvent, for example toluene or in a 1:1 mixture of acetonitrile and dioxane, preferably at −10 to 0° C. or at RT.

A compound of formula VII can be prepared for example by reacting a compound of formula VIII 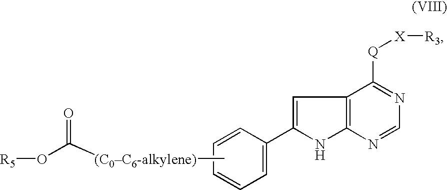
wherein R₅ is lower alkyl, especially methyl or ethyl, and R₃, Q and X have the meanings as defined for a compound of formula I, with lithium aluminium hydride, in an inert solvent, especially ethers, e.g. cyclic ethers such as tetrahydrofuran, preferably at the reflux temperature of the solvent employed. Alternatively, a compound of formula VII may be prepared by reacting a compound of formula VIII with diisobutyl-aluminium hydride, in an inert solvent, for example in tetrahydrofuran or in a 1:1 mixture of dichloromethane and dioxane, preferably at RT.

A compound of formula VIII wherein Q is —NH— can be prepared for example by reacting a compound of formula IX 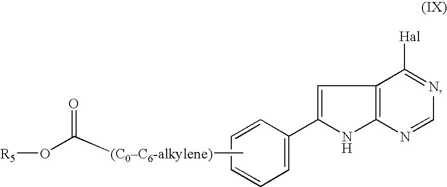
wherein Hal is halogen, preferably chloro, and R₅ is as defined above for a compound of formula VIII, with a compound of the formula H₂N—X—R₃, wherein R₃ and X have the meanings as defined for a compound of formula I, (i) in a suitable solvent such as alcohols, especially lower alcohols such as n-butanol, preferably at the boiling temperature of the solvent employed or (ii) under catalytic conditions.

A compound of formula VIII wherein Q is —O— can be prepared for example by reacting a compound of formula IX, which is preferably N-protected in the pyrrolo-pyrimidine moiety, with a compound of the formula HO—X—R₃, wherein R₃ and X have the meanings as defined for a compound of formula I, in a suitable inert solvent such as N,N-dimethylformamide and in the presence of a base such as potassium carbonate, at elevated temperatures, preferably at around 100° C.

Alternatively, the carboxylic acid ester of a compound of formula IX may first be reduced to the corresponding alcohol, e.g. under conditions described above for the preparation of a compound of formula VII, and then either be reacted with a compound of the formula H₂N—X—R₃, e.g. under conditions described above for the preparation of a compound of formula VIII wherein Q is —NH—, or be reacted with a compound of the formula HO—X—R₃, e.g. under conditions described above for the preparation of a compound of formula VIII wherein Q is —O—.

A compound of formula I, or a pharmaceutically acceptable salt thereof, can be used in pharmaceutical compositions known as such. Compositions for enteral administration, such as nasal, buccal, rectal or, especially, oral administration, and for parenteral administration, such as intravenous, intramuscular or subcutaneous administration, to warm-blooded animals, especially humans, are especially preferred. The compositions contain the active ingredient alone or, preferably, together with a pharmaceutically acceptable carrier. The dosage of the active ingredient depends upon the disease to be treated and upon the species, its age, weight, and individual condition, the individual pharmacokinetic data, and the mode of administration. The pharmaceutical compositions comprise from approximately 1% to approximately 95% active ingredient, single-dose administration forms comprising in the preferred embodiment from approximately 20% to approximately 90% active ingredient and forms that are not of single-dose type comprising in the preferred embodiment from approximately 5% to approximately 20% active ingredient. Unit dose forms are, for example, coated and uncoated tablets, ampoules, vials, suppositories or capsules. Examples are capsules containing from about 0.05 g to about 1.0 g of active substance.

The pharmaceutical compositions of the present invention are prepared in a manner known per se, for example by means of conventional mixing, granulating, coating, dissolving or lyophilising processes.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

In one preferred embodiment of the invention, the solid tumor disease to be treated is renal cell cancer. In another preferred embodiment of the invention, the solid tumor disease to be treated is NSCLC. In a further preferred embodiment of the invention, the solid tumor disease to be treated is selected from skin squamous cell carcinoma and head and neck squamous cell carcinoma. In another preferred embodiment of the invention, the solid tumor disease is anorectal cancer, especially anorectal adenocarcinoma and squamous cell carcinoma of the anal canal and margin and metastasis thereof.

In one embodiment the present invention relates to a treatment regimen whereby the 7H-pyrrolo[2,3-d]pyrimidine derivative is administered to the human subject less frequently than on a daily basis. In particular, the present invention relates to a treatment regimen whereby over at least a three week period, the 7H-pyrrolo[2,3-d]pyrimidine derivative is administered on only about 40% to about 71% of the days. In such embodiment, specifically, the present invention relates to a method of treating a human subject with a 7H-pyrrolo[2,3-d]pyrimidine derivative, which comprises administering such pyrimidine derivative to the human subject from three to five times in each seven day period for a period of three weeks or longer, more specifically, three or four times a week on alternate days for a period of three weeks or longer. In a specific embodiment, the 7H-pyrrolo[2,3-d]pyrimidine derivative is administered three times each week on alternate days, for example, on Monday, Wednesday and Friday of each week, for at least three weeks. Thus, the 7H-pyrrolo[2,3-d]pyrimidine derivative is administered every other day until three doses are given and the next dose is administered at the beginning of the following week. Preferably, such dosage regimen is carried out through at least four or more weeks, for example 4, 5, 6, 7 or 8 weeks. Alternatively, the 7H-pyrrolo[2,3-d]pyrimidine derivative is administered daily for a period of one to three weeks, e.g. two weeks, followed by a period of one to three weeks, e.g. two weeks without administering the compound to the patient.

Thus, the present invention relates especially to a method of treating a solid tumor disease as defined herein, which comprises administering a pharmaceutically effective amount of (R)-6-(4-hydroxy-phenyl)-4-[(1-phenyl-ethyl)-amino]-7H-pyrrolo[2,3-d]pyrimidine, or a salt thereof, to a human subject, preferably three or four times a week on alternate days, more preferably three times a week on alternate days, for a period of three weeks or longer.

The inventive dosage regimen applies to the use of 7H-pyrrolo[2,3-d]pyrimidine derivative, for example, PKI166, alone, or as part of a combination treatment therapy wherein it is co-administered with one or more additional pharmaceutical products useful for treating tumors, especially cancerous tumors. For purposes of this application co-administered means that the patient is treated with both drugs according to the proper schedule for each, but not necessarily that both drugs are administered together at the same time. Thus, the 7H-pyrrolo[2,3-d]pyrimidine derivative, may be administered alone or in combination with other anticancer agents, e.g. in accordance with the present inventive dosage regimen.

The 7H-pyrrolo[2,3-d]pyrimidine derivative is advantageously administered to the human subject at a pharmaceutically effective dosage in the range of from about 50 mg to about 2000 mg on days when the 7H-pyrrolo[2,3-d]pyrimidine derivative is administered. When PKI166 is employed, the preferred effective dose is in the range from about 50 mg to about 2000 mg, for example, about 450 mg to about 1500 mg doses or about 500 mg to about 1200 mg doses.

The 7H-pyrrolo[2,3-d]pyrimidine derivative is administered to the subject by methods known in the art for administering pharmaceutical products, for example, orally, rectally or parenterally, preferably orally as a tablet or capsule formulation. Especially, the 7H-pyrrolo[2,3-d]pyrimidine derivative can be administered as described in WO 97/02266.

The effect of a 7H-pyrrolo[2,3-d]pyrimidine derivative against the tumor types mentioned herein can be demonstrated, e.g., in suitable tumor models utilising cells lines, e.g. models utilising the cell lines NCI-H529 SCC (lung) or orthotopic 253J B-V (bladder).

The following Examples illustrate the invention described above; they are not, however, intended to limit the scope of the invention in any way. The beneficial effects of the 7H-pyrrolo[2,3-d]pyrimidine derivatives can also be determined by other test models known as such to the person skilled in the pertinent art.

### EXAMPLES

### Example 1

### {6-[4-(4-Methyl-piperazin-1-ylmethyl)-phenyl]-7H-pyrrolo[2,3-d]pyrimidin-4-yl}-((R)-1-phenyl-ethyl)-amine

A mixture of 10.8 g (30 mmol) [6-(4-chloromethyl-phenyl)-7H-pyrrolo[2,3-d]pyrimidin-4-yl]-((R)-1-phenyl-ethyl)-amine in 450 ml DMF is treated with 6.8 ml (63 mmol) N-methyl-piperazine and 20.7 g (150 mmol) anhydrous potassium carbonate and the mixture heated to 65° C. for 1 hour. The reaction mixture is cooled and the inorganic salts removed by filtration (Hyflo Super Cel®; Fluka, Buchs, Switzerland). The DMF is evaporated under reduced pressure and the residue purified through flash chromatography using first dichloro-methane/ethanol 9:1 and then dichloromethane/ethanol 9:1 plus 1% conc. ammonia. Crystallization of the pure fractions from THF (20 ml) and hexanes (80 ml) gives the title compound; m.p. 248-250° C.; MS-ES⁺: (M+H)⁺=427.

Step 1.1: 4-[4-((R)-1-Phenyl-ethylamino)-7H-pyrrolo[2,3-d]pyrimidin-6-yl]-benzoic acid ethyl ester

1.8 g (6 mmol) 4-(4-chloro-7H-pyrrolo[2,3-d]pyrimidin-6-yl)-benzoic acid ethyl ester (WO 97/02266) are suspended in 40 ml n-butanol and treated with 1.5 ml (12 mmol) (R)-phenethylamine. The mixture is heated to 145° C. under stirring. After 3 h a clear brown solution is obtained which is treated with a second portion of (R)-phenethylamine (0.75 ml, 6 mmol). After stirring for additional 2 h the reaction mixture is cooled in an ice bath and the title compound filtered and washed with cold n-butanol and ether; m.p. 288-290° C.

Step 1.2: {4-[4-((R)-1-Phenyl-ethylamino)-7H-pyrrolo[2,3-d]pyrimidin-6-yl]-phenyl}-methanol

570 mg (15 mmol) lithium aluminum hydride are suspended in 150 ml dry THF at RT. 1.23 g (3-mmol) 4-[4-((R)-1-phenyl-ethylamino)-7H-pyrrolo[2,3-d]pyrimidin-6-yl]-benzoic acid ethyl ester are added and the mixture heated to reflux for 1 h. The mixture is cooled in an ice bath and treated sequentially with water (0.57 ml), 15% sodium hydroxide solution (0.57 ml) and water (1.71 ml). The solid aluminum complex is removed by filtration (Hyflo Super Cel®; Fluka, Buchs, Switzerland), the filtrate dried over sodium sulfate evaporated. The residue is suspended in water, filtered and dried to give the title compound; m.p. >300° C.; R_{f} (dichloromethane/ethanol 9:1 plus 1% conc. ammonia)=0.43.

Step 1.3: [6-(4-Chloromethyl-phenyl)-7H-pyrrolo[2,3-d]pyrimidin-4-yl]-((R)-1-phenyl-ethyl)-amine

A solution of thionyl chloride (25.7 ml, 0.328 mol) in 180 ml of toluene is cooled to −10° C. Solid {4-[4-((R)-1-phenyl-ethylamino)-7H-pyrrolo[2,3-d]pyrimidin-6-yl]-phenyl}-methanol (11.3 g, 0.0328 mol) is added in 8 portions over a range of 1 h. The temperature is then increased slowly to 0° C. and the mixture stirred for 2 h. The cold reaction mixture is filtered and the solid washed with toluene and ether. The crude product is suspended in water and treated with saturated sodium bicarbonate solution until the mixture turns basic. The mixture is stirred well for about 10 min and filtered. The solid is thoroughly washed with water and dried under reduced pressure to give the title compound; m.p. >320° C.; R_{f} (dichloromethane/ethanol 9:1)=0.46.

### Examples 2-10

The following Examples are synthesized from [6-(4-chloromethyl-phenyl)-7H-pyrrolo[2,3-d]pyrimidin-4-yl]-((R)-1-phenyl-ethyl)-amine using an analogous procedure described in Example 1:

| Exam- | | m.p. |
|---|---|---|
| ple | Name | [° C. ] |
| 2 | [6-(4-Diethylaminomethyl-phenyl)-7H-pyrrolo[2,3- | 246-248 |
| | d]pyrimidin-4-yl]-((R)-1-phenyl-ethyl)-amine | |
| 3 | {6-[4-(4-Ethyl-piperazin-1-ylmethyl)-phenyl]-7H- | 245-247 |
| | pyrrolo[2,3-d]pyrimidin-4-yl}-((R)-1-phenyl-ethyl)- | |
| | amine | |
| 4 | ((R)-1-Phenyl-ethyl)-[6-(4-pyrrolidin-1-ylmethyl- | 254-256 |
| | phenyl)-7H-pyrrolo[2,3-d]pyrimidin-4-yl]-amine | |
| 5 | [6-(4-Dimethylaminomethyl-phenyl)-7H- | 241-243 |
| | pyrrolo[2,3-d]pyrimidin-4-yl]-((R)-1-phenyl-ethyl)- | |
| | amine | |
| 6 | ((R)-1-Phenyl-ethyl)-[6-(4-piperidin-1-ylmethyl- | 246-248 |
| | phenyl)-7H-pyrrolo[2,3-d]pyrimidin-4-yl]-amine | |
| 7 | [6-(4-Morpholin-4-ylmethyl-phenyl)-7H- | 263-265 |
| | pyrrolo[2,3-d]pyrimidin-4-yl]-((R)-1-phenyl-ethyl)- | |
| | amine | |
| 8 | {6-[4-(3,5-Dimethyl-piperazin-1-ylmethyl)-phenyl]- | 208-210 |
| | 7H-pyrrolo[2,3-d]pyrimidin-4-yl}-((R)-1-phenyl- | |
| | ethyl)-amine | |
| 9 | (6-{4-[(2-Morpholin-4-yl-ethylamino)-methyl]- | 222-224 |
| | phenyl}-7H-pyrrolo[2,3-d]pyrimidin-4-yl)-((R)-1- | |
| | phenyl-ethyl)-amine | |
| 10 | ((R)-1-Phenyl-ethyl)-(6-{4-[(tetrahydro-pyran-4- | 253-255 |
| | ylamino)-methyl]-phenyl}-7H-pyrrolo[2,3- | |
| | d]pyrimidin-4-yl)-amine | |

### Example 11

A human patient suffering from renal cell cancer is treated for a period of 16 weeks in 4 cycles consisting of administration of 600 mg of PKI166 daily for two weeks followed by 2 weeks without administering the drug. During such 16 weeks a stable disease is observed.

### Example 12

A human patient suffering from renal cell cancer is treated for a period of 16 weeks with 400 mg of PKI166 on Monday, Wednesday and Friday of each week. During such 16 weeks a stable disease is observed.

### Example 13

A human patient suffering from NSCLC is treated for a period of 8 weeks with 450 mg/day of PKI166 except on day 2, 16 and 30 on which days no drug is applied. After such treatment a partial response is observed.

1-14. (canceled)

15: The use of a 7H-pyrrolo[2,3-d]pyrimidine derivative of the following formula (I′) 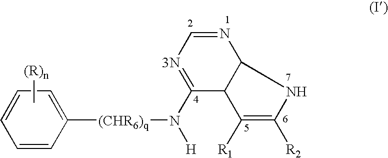
wherein
q is 0 or 1;
n is from 1-3 when q is 0, or n is from 0-3 when q is 1;
R is halogen, lower alkyl, hydroxy, lower alkanoyloxy, lower afkoxy, carboxy, lower alkoxycarbonyl, carbamoyl, N-lower alkyl-carbamoyl, N,N-di-lower alkyl-carbamoyl, cyano, amino, lower alkanoylamino, lower alkylamino, N,N-di-lower alkylamino or tri-fluoromethyl, it being possible when several radicals R are present in the molecule for those radicals to be identical or different;
a) R₁ and R₂ are each independently of the other
α) phenyl substituted by carbamoyl-methoxy, carboxy-methoxy, benzyloxycarbonyl-methoxy, lower alkoxycarbonyl-methoxy, phenyl, amino, lower alkanoylamino, lower alkylamino, N,N-di-lower alkylamino, hydroxy, lower alkanoyloxy, carboxy, lower alkoxycarbonyl, carbamoyl, N-lower alkyl-carbamoyl, N,N-di-lower alkyl-carbamoyl, cyano or by nitro;
β) hydrogen with the proviso that R₁ and R₂ are not hydrogen simultaneously;
γ) unsubstituted or halo- or lower alkyl-substituted pyridyl;
δ) N-benzyl-pyridinium-2-yl, naphthyl, cyano, carboxy, lower alkoxycarbonyl, carbamoyl, N-lower alkyl-carbamoyl, N,N-di-lower alkyl-carbamoyl, N-benzyl-carbamoyl, formyl, lower alkanoyl, lower alkenyl, lower alkenyloxy; or
ε) lower alkyl substituted by
εα) halogen, amino, lower alkylamino, piperazino, di-lower alkylamino,
εβ) phenylamino that is unsubstituted or substituted in the phenyl moiety by halogen, lower alkyl, hydroxy, lower alkanoyloxy, lower alkoxy, carboxy, lower alkoxycarbonyl, carbamoyl, N-lower alkyl-carbamoyl, N,N-di-lower alkyl-carbamoyl, cyano, amino, lower alkanoylamino, lower alkylamino, N,N-di-lower alkylamino or by trifluoromethyl,
εγ) hydroxy, lower alkoxy, cyano, carboxy, lower alkoxycarbonyl, carbamoyl, N-lower alkyl-carbamoyl, N,N-di-lower alkyl-carbamoyl, mercapto or
εδ) by a radical of the formula R₃—S(O)m-, wherein R₃ is lower alkyl and m is 0, 1 or 2; or
b) when q is 0, one of the radicals R₁ and R₂ is unsubstituted lower alkyl or unsubstituted phenyl and the other of the radicals R₁ and R₂ has one of the meanings given above in paragraph a) with the exception of hydrogen; or
c) when q is 0 and R is carboxy, carbamoyl, N-lower alkyl-carbamoyl, N,N-di-lower alkyl-carbamoyl or lower alkanoyl-amino, R₁ and R₂ together are C₄-C₁₀-1,4-alkadienylene substituted by amino, lower alkanoylamino, lower alkylamino, N,N-di-lower alkylamino, nitro, halogen, hydroxy, lower alkanoyloxy, carboxy, lower alkoxycarbonyl, carbamoyl, N-lower alkyl-carbamoyl, N,N-di-lower alkyl-carbamoyl or by cyano, or are aza-1,4-alkadienylene having up to 9 carbon atoms; or
d) when q is 1, R₁ and R₂ are each independently of the other unsubstituted lower alkyl or unsubstituted phenyl or have one of the meanings given above in paragraph a), and R₆ is hydrogen, lower alkyl, lower alkoxycarbonyl, carbamoyl, N-lower alkyl-carbamoyl or N,N-di-lower alkyl-carbamoyl, with the exception of the compound of formula (I), wherein n is 0, q is 1, R₁ and R₆ are each hydrogen and R₂ is methyl, the compound of formula (I′), wherein n is 2, q is 1, R₁ and R₆ are each hydrogen, R₂ is methyl and R is 3,4-methoxy and the compounds of formula (I′), wherein n is 1, q is 0, R₁ is 3-pyridyl, 4-cyanophenyl or 4-hydroxyphenyl, R₂ is hydrogen and R is fluoro, and wherein the prefix “lower” denotes a radical having up to and including a maximum of 7 carbon atoms;
or a pharmaceutically acceptable salt thereof, for the manufacture of a medicament for the treatment of renal carcinoma.

16: The use according to claim 15, wherein the 7H-pyrrolo[2,3-d]pyrimidine derivative of formula (I′) is (R)-6-(4-hydroxy-phenyl)-4-[(1-phenyl-ethyl)-amino]-7H-pyrrolo[2,3-d]pyrimidine.

17: A method for the treatment of patient suffering from renal carcinoma comprising administering a pharmaceutically effective amount of a 7H-pyrrolo[2,3-d]pyrimidine derivative of formula (I′) according to claim 15, or a pharmaceutically acceptable salt thereof, to the patient.

18: The method of claim 17, wherein the pharmaceutically effective dose is in the range from about 50 mg to about 2000 mg.

19: A method of inhibiting metastatic growth in a patient with a renal carcinoma which comprises administering a pharmaceutically effective amount of a 7H-pyrrolo[2,3-d]pyrimidine derivative of formula (I′) according to claim 15, or a pharmaceutically acceptable salt thereof, to the patient.

20: Use of a 7H-pyrrolo[2,3-d]pyrimidine derivative of formula (I) 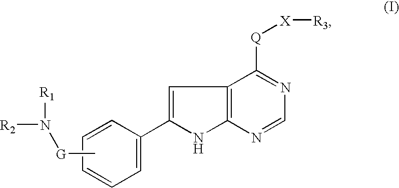
wherein
R₁ and R₂ are each independently of the other hydrogen, unsubstituted or substituted alkyl or cycloalkyl, a heterocyclic radical bonded via a ring carbon atom, or a radicat of the formula R₄—Y—(C═Z)—, wherein R₄ is unsubstituted, mono- or disubstituted amino or a heterocyclic radical, Y is either not present or lower alkyl and Z is oxygen, sulfur or imino, with the proviso that R₁ and R₂ are not both hydrogen; or
R₁ and R₂, together with the nitrogen atom to which they are attached, form a heterocyclic radical;
R₃ is a heterocyclic radical or an unsubstituted or substituted aromatic radical;
G is C₁-C₇-alkylene, —C(═O)—, or C₁-C₆-alkylene-C(═O)—, wherein the carbonyl group is attached to the NR₁R₂ moiety;
Q is —NH— or —O—, with the proviso that Q is —O— if G is —C(═O)— or C₁-C₆-alkylene-C(═O)—; and
X is either not present or C₁-C₇-alkylene, with the proviso that a heterocyclic radical R₃ is bonded via a ring carbon atom if X is not present;
or a pharmaceutically acceptable salt thereof, for the manufacture of a medicament for the treatment of carcinoma of the bladder, renal carcinoma, squamous cell carcinoma of the skin, tumors of the gastrointestinal tract, mesothelioma, esophageal tumors, stomach cancer, small-bowel tumors and large-bowel tumors such as polyps of the colon and rectum and anorectal cancer.

21: A method for the treatment of patients suffering from a solid tumor disease selected from carcinoma of the bladder, renal carcinoma, squamous cell carcinoma of the skin, tumors of the gastrointestinal tract, mesothelioma, esophageal tumors, stomach cancer, small-bowel tumors and large-bowel tumors, such as polyps of the colon and rectum and anorectal cancer, comprising administering to the patient a pharmaceutically effective amount of a 7H-pyrrolo[2,3-d]pyrimidine derivative of formula (I) according to claim 20 or a pharmaceutically acceptable salt of said compound.

22: The method of claim 21, wherein the pharmaceutically effective dose is in the range from about 50 mg to about 2000 mg.

23: A method of inhibiting metastatic growth in a patient with a solid tumor disease selected from carcinoma of the bladder, renal carcinoma, squamous cell carcinoma of the skin, tumors of the gastrointestinal tract, mesothelioma, esophageal tumors, stomach cancer, small-bowel tumors and large-bowel tumors, such as polyps of the colon and rectum and anorectal cancer which comprises administering to the patient a pharmaceutically effective amount of a 7H-pyrrolo[2,3-d]pyrimidine derivative of formula (I) according to claim 20 or a salt of the said compounds, for the treatment of carcinoma of the bladder, renal carcinoma, squamous cell carcinoma of the skin, tumors of the gastrointestinal tract, mesothelioma, esophageal tumors, stomach cancer, small-bowel tumors and large-bowel tumors, such as polyps of the colon and rectum and anorectal cancer.
